(19) United States
(12) Patent Application Publication
(10) Pub. No.: US 2011/0009804 A1
(43) Pub. Date: Jan. 13, 2011

(54) Method and System for Delivering a Medicant

(75) Inventor: Robert J. Behnke, Erie, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/499,187

(22) Filed: Jul. 8, 2009

(51) Int. Cl. A61F 7/08; A61M 31/00; A61N 1/04

(52) U.S. Cl. 604/20; 607/102; 604/500

(57) ABSTRACT

The present disclosure provides a method for administering chemotherapy to a patient. The method includes heating target tissue to a first temperature to cause hyperthermia, wherein the temperature is in the range of about 40° C. to about 45° C. The method includes allowing the body to compensate for the hyperthermia by causing hyperfusion and increased blood flow to the target tissue. The method includes administering a chemotherapeutic drug adjacent the target tissue and allowing the chemotherapeutic drug to perfuse the target tissue; and heating tissue to a second temperature to ablate the target tissue.

### BACKGROUND

1. Technical Field

The present disclosure relates to a method and system for delivering a medicant and, more particularly, to a method and system for delivering chemotherapy to a cancer region of a patient.

2. Description of Related Art

Chemotherapy treatment, in its most general sense, refers to treatment of disease by chemicals that kill cells (both cancer and non-cancer cells) In conventional usage, chemotherapy refers to antineoplastic drugs used to treat cancer or the combination of these drugs into a cytotoxic standardized treatment regimen. Chemotherapeutic drugs may cause damage to cells or cause cells to undergo apoptosis (so-called “programmed cell death”). Conventionally, chemotherapy is used to treat tumors by impairing mitosis (cell division). More particularly, chemotherapy acts by killing cells that divide rapidly (one of the main properties of cancer cells).

In certain instances, chemotherapy may become less effective due to the cancer cells becoming more resistant to the chemotherapy treatments. For example, if a tumor is more developed it becomes more resistant to chemotherapy because the center of the tumor doesn't have cell division occurring as rapidly; this may have a deleterious effect to a patient. That is, because chemotherapy is typically released through the entire body, there is the likelihood that the chemotherapy will affect cells that divide rapidly under normal circumstances, such as, for example, cells in bone marrow, which may result in myclosuppression (decreased production of blood cells), cells in the digestive tract, which may result in mucositis (inflammation of the lining of the digestive tract), and cells in hair follicles, which may result in alopecia (hair loss).

### SUMMARY

The present disclosure provides a system for treating disease. The system includes one or more electrosurgical generators providing a source of electrosurgical energy and capable of operating in one or more modes. A first mode is configured to deliver electrosurgical energy capable of heating tissue to a first temperature, wherein the first temperature is in the range from about 40° C. to about 45° C. and a second mode is configured to deliver electrosurgical energy capable of heating tissue to a second temperature, wherein the second temperature is sufficient to cause necrosis to tissue. The system includes a microwave antenna assembly adapted to connect to the electrosurgical generator and configured to heat a region of tissue to the first and second temperatures. A control system is in operative communication with the electrosurgical generator and configured to control an output of the electrosurgical generator. The system also includes one or more sensors in operative communication with the control system and configured to monitor a temperature adjacent a tissue site.

The present disclosure provides a method for administering chemotherapy to a patient. The method includes heating target tissue to a first temperature to cause hyperthermia, wherein the temperature is in the range of about 40° C. to about 45° C. The method includes allowing the body to compensate for the hyperthermia by causing hyperfusion and increased blood flow to the target tissue. The method includes administering a chemotherapeutic drug adjacent the target tissue and allowing the chemotherapeutic drug to perfuse the target tissue; and heating tissue to a second temperature to ablate the target tissue.

The present disclosure also provides a method for administering chemotherapy to a patient. The method includes providing one or more electrosurgical generators capable of operating in one or more modes. A first mode configured to deliver electrosurgical energy capable of heating tissue to a first temperature and a second mode configured to deliver electrosurgical energy capable of heating tissue to a second temperature. The providing step includes providing a microwave antenna assembly adapted to connect to the electrosurgical generator and configured to heat a region of tissue to the first and second temperatures. The providing step includes providing a control system in operative communication with the electrosurgical generator and configured to control an output of the at least one electrosurgical generator. The providing step also includes providing one or more sensors in operative communication with the control system and configured to monitor a temperature adjacent a tissue site. A step of the method includes heating tissue at a tumor site to the first temperature such that tissue at the tumor site becomes hyperthermic. A step of the method includes administering a chemotherapeutic drug to tissue adjacent the tissue site.

### BRIEF DESCRIPTION OF THE DRAWING

Various embodiments of the present disclosure are described hereinbelow with references to the drawings, wherein:

FIG. 1 is a perspective view of an energy-based delivery system according to an embodiment of the present disclosure;

FIG. 2 is a simplified block diagram of the energy-based ablation system depicted in FIG. 1 being used on a patient;

FIG. 3 is a detailed block diagram illustrating various modules of the electrosurgical generator depicted in FIG. 2;

FIG. 4 is a simplified block diagram of an energy based ablation system according to an alternate embodiment of the present disclosure; and

FIG. 5 is a flow chart illustrating a method for treating disease according to an embodiment of the present disclosure.

### DETAILED DESCRIPTION

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

A method and system according to the present disclosure is configured to increase, and more effectively, delivery one or more types of chemotherapeutic drugs associated with chemotherapy to a cancer region. The method and system of the present disclosure delivers electrosurgical energy to heat a cancer region, e.g., a tumor and/or surrounding tissue until hyperthermia occurs.

More particularly, blood temperature inside a human body is normally between 34° to 37° C. As temperature of tissue rises between 40° to 45° C., a body will try to compensate by hyperfusion (increase blood flow) to the tissue. As tissue becomes more perfused, the arteries become dilated and, in a sense, “open up” the tissue to increase the blood flow thereto. As disclosed herein, hyperfusion may be implemented for the purpose of dilating a developed tumor for better treatment. More particularly, in an instance where a patient is undergoing chemotherapy, heating and, thus, dilating a developed tumor such that the tumor becomes perfused, increases blood flow to the tumor, which, in turn, results in an increase in an amount of chemotherapy delivered to the tumor and/or surrounding area.

Concentrating and/or delivering a volume of chemotherapy to a specific cancer region (e.g., a tumor) reduces the overall amount of chemotherapy needed by a patient, which, in turn, may reduce and/or eliminate one or more of the aforementioned side effects of chemotherapy. With this purpose in mind, the present disclosure includes one or more types of energy based systems (EBS) for delivering electrosurgical energy to a tissue site and subsequently heating tissue at (or adjacent) the tissue site to within a predetermined temperature range, e.g.) 40° to 45° C.

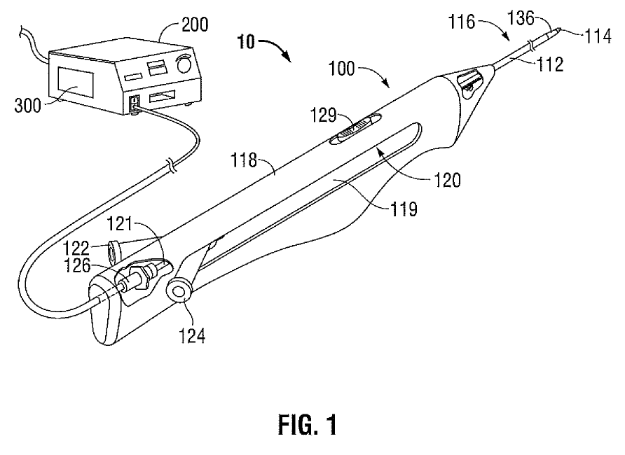

With reference to FIG. 1, a suitable EBS for delivering electrosurgical energy to a tissue site is shown designated 10. In the embodiment illustrated in FIG. 1, EBS 10 is a microwave heating and/or ablation system 10 (system 10). System 10 includes a microwave antenna assembly 100 adapted to connect to a source of electrosurgical energy. In the embodiment illustrated in FIG. 1, the source of electrosurgical energy is an electrosurgical generator 200 (generator 200). System 10 includes a control system 300 in operative communication with the electrosurgical generator 200 for performing an electrosurgical procedure (e.g., heating and/or ablating procedure).

With continued reference to FIG. 1, microwave antenna assembly 100 is shown. In the embodiment illustrated in FIG. 1, microwave antenna assembly 100 includes an introducer 116 having an elongated shaft 112, a conductive member 114 slidably disposed within elongated shaft 112, a thermal sensor or detector 136 for detecting tissue temperature, a cooling assembly 120 having a cooling sheath 121, a cooling fluid supply 122 and a cooling fluid return 124, and an electrosurgical energy connector 126. Connector 126 is configured to connect the microwave antenna assembly 100 to an electrosurgical power generating source 200, e.g., a generator 200 or other suitable source of radio frequency energy and/or microwave energy, and supplies electrosurgical energy to the distal portion of the microwave antenna assembly 100. A selector 129 is positioned on the proximal end of a handle 118 and connects to the electrosurgical energy delivery source 200. Selector 129 provides a means for a clinician to select the energy type, e.g., radio frequency or microwave, the energy delivery mode, e.g., bipolar, monopolar, and the mode of operation, e.g., thermal treatment mode and/or thermal treatment in conjunction with an ablation cycle mode.

For a more detailed description of a microwave antenna assembly 100 and/or operative components associated therewith, reference is made to commonly-owned United States Patent Publication No. 2008/0082093 filed Sep. 29, 2006.

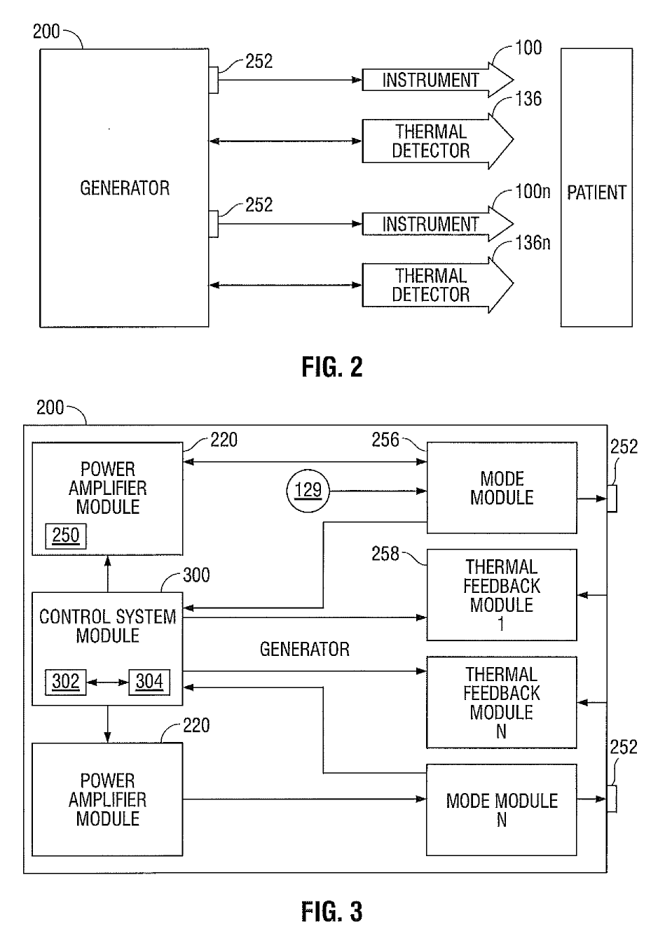

With reference again to FIG. 1, an illustrative embodiment of a generator 200 and system 300 are shown. Generator 200 operatively and selectively connects to one or more microwave antenna assemblies 100 for performing an electrosurgical procedure, e.g., heating and/or ablating tissue, and a corresponding thermal detector 136 (see FIGS. 1 and 2). In the embodiment illustrated in FIG. 2, generator 200 is shown operatively connected to “n” number of microwave antenna assemblies 100, 100n, respectively, and “n” number of corresponding thermal detectors 136 136n, respectively.

So as not to obscure the present disclosure with redundant information, generator 200 and control system 300 will be described in terms of use with a single microwave antenna assembly 100.

Generator 200 may be configured for monopolar and/or bipolar modes of operation. Generator 200 includes suitable components, parts, and/or members needed for a control system 300 to function as intended. Generator 200 generates electrosurgical energy, e.g., RF (radio frequency), microwave, or other electrosurgical energy.

With reference to FIG. 3, a detailed block diagram of the operative components associated with generator 200 is shown. One or more power amplifier modules 220 generates microwave energy and includes a power supply 250 for generating energy and an output stage 252 (shown for illustrative purposes external the generator 200) that modulates the energy provided to the delivery device(s), such as the conductive member 114 of microwave assembly 100, for delivery of the electrosurgical energy to a patient. Power supply 250 may be a high voltage DC or AC power supply for producing electrosurgical current, where control signals generated by the system 300 adjust parameters of the voltage and current output, such as magnitude and frequency. Power amplifier module 220 is in operative communication with a corresponding mode module 256 and control system 300. Power amplifier module 220 may be digital and/or analog circuitry that can receive instructions from and provide status to a processor 302 of control system 300 (via, for example, a digital-to-analog or analog-to-digital converter). Power amplifier module 220 can amplify, filter, and digitally sample return signals received by thermal detector 136 via control system 300. In the embodiment illustrated in FIG. 3, power amplifier module 220 receive inputs from the control system 300 and outputs a corresponding output signal to mode module 256.

Mode module 256 is in operative communication with control system 300 and power amplifier module 220. Mode module 256 may be in operative communication with selector 129. In this instance, a user may select system 10 to function in one or more modes of operation. More particularly, a mode of operation may include a treatment mode. In this instance, one or S more of the microwave ablation assemblies 100 may be used to heat a specific region of tissue, e.g., a tumor to hyperthermia. More particularly, the tumor, or portions thereof, may be heated to a temperature within a range of 40° to 45° C. As previously noted, heating tissue to within this range of temperatures does not cause tissue ablation. In an embodiment, a mode of operation may include an ablation cycle. In this instance, after the thermal treatment mode is used to ensure that the chemotherapy was delivered to the tumor, the ablation cycle may be used to ensure that tumor cells were heated to cell necrosis; giving a better chance for total treatment of the tumor and surrounding area.

A thermal feedback module 258 is in operative communication with a corresponding thermal detector 136 and senses electromagnetic, electrical, and/or physical parameters or properties at the operating site and communicates with the control system 300 to regulate the output electrosurgical energy at the conductive member 114. The thermal sensor module 258 may be configured to measure, i.e., “sense”, various electromagnetic, electrical, physical, and/or electromechanical conditions, such as at or proximate the operating site, including: tissue impedance, tissue temperature, and so on. For example, a thermal detector 136 in operative communication with the thermal detector module 258 may measure one or more of these conditions continuously or in real-time such that the control system 300 can continually modulate the electrosurgical output. Thermal feedback module 258 may be used to ensure that a specific tissue region is properly heated without causing tissue necrosis. More particularly, thermal feedback module 258 may include a feedback loop that indicates when tissue has been properly heated based upon one or more of the following parameters: tissue temperature, tissue impedance at the tissue site, change in impedance of the tissue over time and/or changes in the power or current applied to the tissue over time.

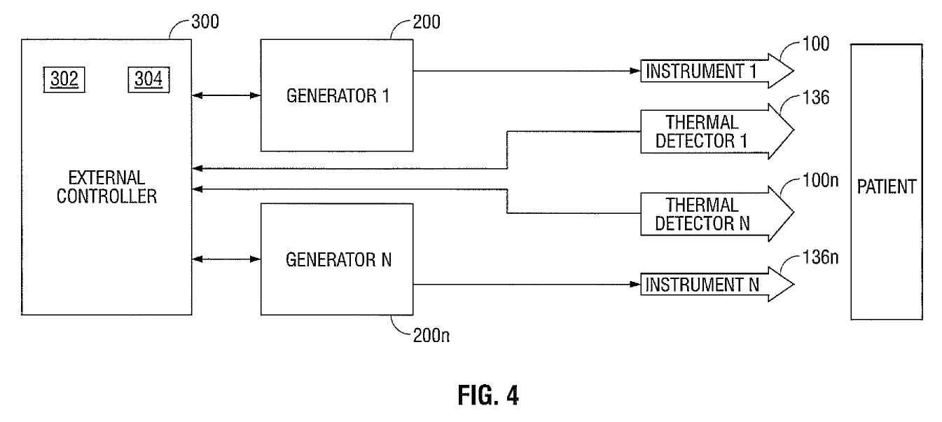

Control system 300 may be coupled to the power amplifier module 220 by connections that may include wired and/or wireless connections for providing the control signals to the power amplifier module 220. In the embodiment illustrated in FIGS. 1-3, control system 300 is housed in generator 200. In this instance, the control system 300 is self contained and can make necessary computations (e.g., mode of operation) internal to generator 200. Alternatively, control system 300 may be located remote to generator 200 (FIG. 4). In this instance, any necessary computations (e.g., mode of operation) are performed externally to generator 200.

Control system 300 is configured to analyze parameters such as, for example, temperature at a tissue site. With this purpose in mind, control system 300 includes one or more processors 302 in operative communication with one or more control modules 304 executable on the processor. The control module 304 may instruct one or more modules, e.g., power amplifier module 220, to transmit electrosurgical energy, which may be in the form of a wave or signal/pulse, via one or more cables to one or more microwave ablation assemblies 100.

The control module 304 processes information and/or signals (e.g., temperature data from thermal detector 136) input to the processor 302 and generates control signals for modulating the electrosurgical energy in accordance with the input information and/or signals. Information may include pre-surgical data (e.g., predetermined temperature ranges) entered prior to the electrosurgical procedure or information entered and/or obtained during the electrosurgical procedure through one or more modules (e.g., thermal feedback module 258) and/or other suitable device.

The control module 304 regulates the generator 200 (e.g., the power supply 250 and/or the output stage 252) which adjusts various parameters (e.g., voltage, current, resistance, etc.). More particularly, the control module 304 regulates the electrosurgical energy in response to thermal feedback information (e.g., information related to tissue condition at or proximate the surgical site). The control module 304 then sends control signals to the power amplifier module 220 for regulating the power supply 250 and/or the output stage 252.

Regulation of certain parameters of the electrosurgical energy may be based on a tissue response such as recognition when a predetermined threshold temperature value is achieved. Recognition of the event may automatically switch the generator 200 to a different mode of operation and subsequently switch the generator 200 back to an original mode after the event has occurred. In embodiments, recognition of the event may automatically switch the generator 200 to a different mode of operation and subsequently shutoff the generator 200.

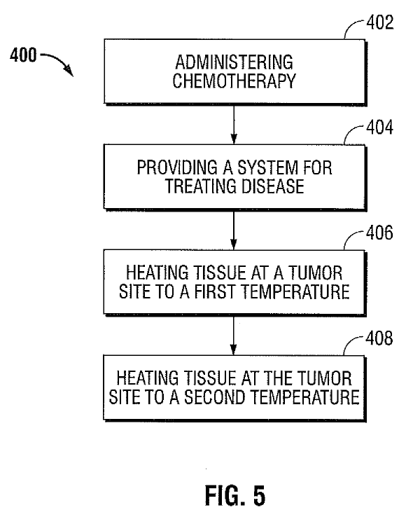

With reference to FIG. 5, a method 400 of use of system 10 for treating disease, e.g., for delivering chemotherapy to a cancer region of a patient according to one embodiment of the disclosure is now described. In the description that follows, it is assumed that chemotherapy has been properly administered to patient (step 402).

A user positions a conductive member 114 associated with system 10 at a tissue site, e.g., tumor site, (see step 404). In an embodiment, a user sets the microwave assembly 100 to a thermal treatment mode via selector 129. Processor 302 instructs control module 304 to transmit electrosurgical energy from the power amplifier module 220 to the conductive element 114 of microwave assembly 100. Data, such as, for example, temperature, impedance and so forth is sensed by thermal detector 136 and transmitted to and sampled by the thermal feedback module 258. The data can be processed by the processor 302 and/or thermal feedback module 258 to determine, for example, when a threshold temperature value (e.g., 40° to 45° C.) has been achieved at the tumor site. The processor 302 can subsequently transmit and/or otherwise communicate the data to the control module 304 such that output power from conductive element 114 may be adjusted accordingly. As noted above, when tissue at the tumor site is heated (step 406) to a temperature of 40° to 45° C., the tumor becomes dilated and perfused, which, in turn causes blood flow to the tumor to increase, which, in turn, results in an increase in an amount of chemotherapy delivered to the tumor and/or surrounding area.

In an embodiment, upon reaching a desired threshold temperature, processor 302 instructs control module 304 to generate electrosurgical energy in response to the processor instructions to conductive element 114 such that a desired tissue effect may be achieved at the tumor site, e.g., tissue ablation (step 408).

From the foregoing and with reference to the various figure drawings, those skilled in the art will appreciate that certain modifications can also be made to the present disclosure without departing from the scope of the same. For example, in an embodiment, a chemotherapeutic drug could be encapsulated inside a heat activated material configured to release only when a tissue temperature is within a predetermined temperature range. In this instance, the chemotherapy would only be initiated in a region (e.g., region where the temperature of tissue is within 40°-45° C.) where it is needed and not the entire body.

In embodiments where system 10 is configured to function in thermal heat mode and not thermal treatment in conjunction with an ablation cycle mode, conductive element 114 does not need to carry as much power as typically required ablating the tissue; this means the antennas could be a lot thinner, such as the 22 gauge, and thus be less intrusive to the patient.

This embodiment of system 10 could be used on patients that are too delicate for the larger antennas or for tumors that are near vital vessel structures that would be damaged during an ablation.

While thermal detector 136 is illustrated operatively coupled to the introducer 116, it is within the purview of the present disclosure to employ a “stand-alone” thermal detector 136 that is in operative communication with the generator 200 and/or control system 300.

It is contemplated that in addition to microwave of RF energy delivery EBS, system 10 may be configured to use various other energy based systems such as, for example, ultrasound systems or piezoelectric devices.

It is contemplated that tissue at the tumor site may be heated to a temperature within the range of 40°-45° C. by any suitable heating mediums. For example, ointments, lotions, compresses, medicant, fluids, gases, and so forth may all be used alone and/or in conjunction with system 10 to heat tissue at the tumor site.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for treating disease, comprising:
at least one electrosurgical generator providing a source of electrosurgical energy and capable of operating in at least two modes, a first mode configured to deliver electrosurgical energy capable of heating tissue to a first temperature, wherein the first temperature is in the range from about 40° C. to about 45° C. and a second mode configured to deliver electrosurgical energy capable of heating tissue to a second temperature, wherein the second temperature is sufficient to cause necrosis to tissue;
a microwave antenna assembly adapted to connect to the electrosurgical generator and configured to heat a region of tissue to the first and second temperatures;
a control system in operative communication with the at least one electrosurgical generator and configured to control an output of the at least one electrosurgical generator; and
at least one sensor in operative communication with the control system and configured to monitor a temperature adjacent a tissue site.

2. A system for treating disease according to claim 1, wherein the microwave antenna assembly is configured to deliver energy in the microwave energy range of about 0.3 GHz to about 300 GHz.

3. A system for treating disease according to claim 1, wherein the microwave antenna assembly is configured to deliver energy in the radio frequency energy range of about 3 Hz to about 300 GHz.

4. A method for administering chemotherapy to a patient comprising the steps of:
heating target tissue to a first temperature to cause hyperthermia, wherein the temperature is in the range of about 40° C. to about 45° C.;
allowing the body to compensate for the hyperthermia by causing hyperfusion and increased blood flow to the target tissue;
administering a chemotherapeutic drug adjacent the target tissue and allowing the chemotherapeutic drug to perfuse the target tissue; and
heating tissue to a second temperature to ablate the target tissue.

5. A method for administering chemotherapy to a patient comprising the steps of:
providing at least one electrosurgical generator capable of operating in at least two modes, a first mode configured to deliver electrosurgical energy capable of heating tissue to a first temperature and a second mode configured to deliver electrosurgical energy capable of heating tissue to a second temperature;
a microwave antenna assembly adapted to connect to the electrosurgical generator and configured to heat a region of tissue to the first and second temperatures;
a control system in operative communication with the at least one electrosurgical generator and configured to control an output of the at least one electrosurgical generator;
at least one sensor in operative communication with the control system and configured to monitor a temperature adjacent a tissue site; and
heating tissue at a tumor site to the first temperature such that tissue at the tumor site becomes hyperthermic; and
administering a chemotherapeutic drug to tissue adjacent the tissue site.

6. A method for administering chemotherapy according to claim 5, further including the step of heating tissue at the tumor site to the second temperature such that a tissue effect is caused at the tumor site.

7. A method for administering chemotherapy according to claim 5, wherein the step of heating tissue to the first temperature includes heating tissue to a temperature from about 40° C. to about 45° C.

8. A method for administering chemotherapy according to claim 6, wherein the step of heating tissue to the second temperature includes heating tissue to a temperature sufficient to cause necrosis.
